(19) United States
(12) Patent Application Publication
(10) Pub. No.: US 2025/0170200 A1
(43) Pub. Date: May 29, 2025

(54) COMPOSITION COMPRISING COFFEE EXTRACT FOR SUPPRESSING POSTPRANDIAL BLOOD SUGAR RISE

(71) Applicants: Yong Ho LEE, Seogwipo-si (KR); PYXIS INC., Daejeon (KR)

(72) Inventor: Yong Ho LEE, Seogwipo-si (KR)

(73) Assignees: Yong Ho LEE, Seogwipo-si (KR); PYXIS INC., Daejeon (KR)

(21) Appl. No.: 18/839,691

(22) PCT Filed: Feb. 17, 2023
(86) PCT No.: PCT/KR2023/002319

§ 371 Date: Sep. 26, 2024

(30) Foreign Application Priority Data
Feb. 20, 2022 (KR) 10-2022-0021815

(51) Int. Cl. A61K 36/74; A61P 3/10

(52) U.S. Cl. CPC A61K 36/742; A61P 3/10; A61K 2236/331

(57) ABSTRACT

The present invention relates to a composition comprising a coffee hot-water extract as an active ingredient for suppressing the postprandial blood sugar rise, wherein the composition can effectively suppress the postprandial blood sugar rise by more strongly inhibiting α-glucosidase and α-amylase activities than typical coffee extracts to suppress the degradation and absorption of polysaccharides, disaccharides, and the like.

### TECHNICAL FIELD

The present invention relates to a composition for suppressing postprandial blood sugar rise, and more specifically, to a composition comprising a coffee extract as an active ingredient for suppressing postprandial blood sugar rise.

### BACKGROUND ART

Recently, according to the American Diabetes Association (ADA), a case where the fasting blood sugar of a human body is 126 mg/dL or more or the blood sugar 2 hours after meal is 200 mg/dl is defined as diabetes.

Blood sugar control is the most important factor in prevention and treatment management of diabetes, and is known as the most important factor that can determine the possibility of causing complications of diabetes. Blood sugar is necessarily and simultaneously with fasting blood sugar, postprandial blood sugar, and glycated hemoglobin. In particular, postprandial blood sugar is a major pathogenesis that causes diabetic vascular complications, and postprandial blood sugar control is a very important factor in preventing the onset of diabetes or diabetic complications in normal groups with good blood sugar control or patients with a short prevalence of diabetes.

Postprandial hyperglycemia promotes an LDL oxidation process, reduces the production and utilization of NO in endothelial cells as well as inhibits FMD, activates the interaction of endothelial cells and leukocytes, and increases various inflammation-inducing and oxidative stress in endothelial cells, thereby reducing endothelial cell function. Since endothelial dysfunction is known as the first stage of cardiovascular disease occurrence and as a marker that can be discovered at the earliest stage, postprandial hyperglycemia may contribute to oxidative stress and endothelial dysfunction, thereby causing vascular complications.

In this regard, Korean Registered Patent No. 10-0996985 discloses a GLP-1 secretion promoter and an inhibitor containing κ-casein as an active ingredient for suppressing a rise in postprandial blood sugar level, and a food product for promoting secretion of GLP-1 and a food product for suppressing a rise in postprandial blood sugar level, in which a milk-derived casein protein is contained and at least 60% by mass of the milk-derived casein protein is K-casein.

In addition, Korean Registered Patent No. 10-0966613 discloses an arginine derivative compound having an effect of suppressing blood sugar rise, Korean Registered Patent No. 10-1155079 discloses a composition for suppressing postprandial blood sugar rise and obesity by using catechin gallate isolated from a green tea extract, and Korean Registered Patent No. 10-0924478 discloses an agent, which is a pharmaceutically acceptable anion exchange resin, for improving postprandial hyperglycemia using cholestymide.

As can be seen from the results of these studies, it is very important to control postprandial hyperglycemia in the onset of diabetes and patients with diabetes, and if therapeutic agents capable of controlling postprandial hyperglycemia are appropriately administered, it may be helpful in preventing diabetes or diabetic complications.

### DISCLOSURE

### Technical Problem

An object of the present invention is to provide a composition capable of controlling postprandial blood sugar which may cause diabetes, controlling blood sugar of diabetic patients, or preventing or reducing vascular complications.

### Technical Solution

To achieve the above object, the present invention provides a composition including a coffee extract as an active ingredient for suppressing postprandial blood sugar rise.

For example, the suppression of postprandial blood sugar rise may mean alleviation of symptoms in diabetic patients by suppressing postprandial blood sugar rise.

According to one embodiment of the present invention, the coffee extract may be a hot-water extract of roasted coffee beans.

According to one embodiment of the present invention, the coffee extract may include a hot-water extract of coffee beans roasted at 200° C. to 250° C. for 30 seconds to 2 minutes and a hot-water extract of coffee beans roasted at 200° C. to 250° C. for 3 to 6 minutes.

In addition, the present invention provides a pharmaceutical composition including a coffee extract as an active ingredient for preventing or treating diabetes.

Further, the present invention provides a food composition including a coffee extract as an active ingredient for preventing or alleviating diabetes.

### Advantageous Effects

According to the present invention, it is possible to provide a composition capable of controlling postprandial blood sugar which may cause diabetes, controlling blood sugar of diabetic patients, or preventing or reducing vascular complications.

In addition, according to the present invention, it is possible to provide a pharmaceutical composition for preventing or treating diabetes, which can be easily and simply produced by using a coffee extract and has excellent medical effects.

### DESCRIPTION OF DRAWINGS

FIG. 1 shows inhibitory activity of a coffee extract against rat intestinal α-glucosidase.

FIG. 2 shows inhibitory activity of caffeic acid, chlorogenic acid, and quinic acid against rat intestinal α-glucosidase.

FIG. 3 shows inhibitory activity of a coffee extract against porcine pancreatic α-amylase.

FIG. 4 shows inhibitory activity of caffeic acid, chlorogenic acid, and quinic acid against porcine pancreatic α-amylase.

FIG. 5 shows a content of a phenol-based compound in a coffee extract.

FIG. 6 shows antioxidant activity of a coffee extract.

FIG. 7 shows inhibition action on postprandial blood sugar rise of a coffee extract by sucrose.

FIG. 8 shows inhibition action on postprandial blood sugar rise of chlorogenic acid by sucrose.

FIG. 9 shows inhibition action on postprandial blood sugar rise of a coffee extract by starch.

FIG. 10 shows inhibition action on postprandial blood sugar rise of chlorogenic acid by starch.

FIG. 11 shows HPLC results of a coffee extract. (a) Mild, (b) medium, (c) Mediumdark, (d) Dark

### MODE FOR INVENTION

Hereinafter, the present invention will be described in detail based on examples. The terms, examples, and the like used in the present invention are merely exemplified to describe the present invention in more detail and to help the understanding of those skilled in the art, and the scope of the present invention should not be construed as being limited thereto.

Unless otherwise defined, the technical terms and scientific terms used in the present invention indicate meanings commonly understood by those skilled in the art to which the present invention pertains.

The present invention relates to a composition for suppressing postprandial blood sugar rise, which includes a coffee extract as an active ingredient.

The coffee extract is a hot-water extract of roasted coffee beans.

As the coffee, coffee beans, roasted coffee beans, and the like may be used, and it is preferable to use roasted coffee beans.

As the roasted coffee beans, coffee beans roasted at 200° C. to 250° C. for 30 seconds to 18 minutes may be used.

In addition, as the roasted coffee beans, at least one of coffee beans (Mild) roasted at 200° C. to 250° C. for 30 seconds to 2 minutes, coffee beans (Medium) roasted at 200° C. to 250° C. for 3 to 6 minutes, coffee beans (Med-dark) roasted at 200° C. to 250° C. for 7 to 10 minutes, and coffee beans (Dark) roasted at 200° C. to 250° C. for 12 to 18 minutes may be used.

According to the present invention, it is preferable to use the coffee beans (Mild) roasted at 200° C. to 250° C. for 30 seconds to 2 minutes, and it is most preferable to use coffee beans roasted at 220° C. for 1 minute.

In addition, according to the present invention, the coffee beans (Mild) roasted at 200° C. to 250° C. for 30 seconds to 2 minutes and the coffee beans (Medium) roasted at 200° C. to 250° C. for 3 to 6 minutes may be used as the roasted coffee beans. In this case, a weight ratio of Mild and Medium is preferably 60 to 80:20 to 40.

In addition, according to the present invention, the coffee beans (Mild) roasted at 200° C. to 250° C. for 30 seconds to 2 minutes, the coffee beans (Medium) roasted at 200° C. to 250° C. for 3 to 6 minutes, and the coffee beans (Dark) roasted at 200° C. to 250° C. for 12 to 18 minutes may be used as the roasted coffee beans. In this case, a weight ratio of Mild, Medium, and Dark is preferably 100:30 to 50:10 to 30.

As the coffee extract, the coffee extract may be obtained by heating and extracting roasted coffee beans with a solvent, filtering the roasted coffee beans, concentrating the resultant under reduced pressure, and then freeze-drying the concentrate.

The heat extraction may be performed by adding 500 to 2,000 parts by weight, 600 to 1600 parts by weight, 700 to 1400 parts by weight, 800 to 1200 parts by weight, 900 to 1100 parts by weight, or 1000 parts by weight of a solvent with respect to 100 parts by weight of roasted coffee beans, and heating and extracting the mixture at 30° C. to 150° C., 100° C. to 140° C., 110° C. to 130° C., 115° C. to 125° C., 120° C. to 123° C., or 121° C. for 10 minutes to 10 hours, 10 minutes to 5 hours, 10 minutes to 1 hour, 10 minutes to 50 minutes, 10 minutes to 40 minutes, 10 minutes to 30 minutes, 10 minutes to 20 minutes, 12 minutes to 18 minutes, or 15 minutes.

According to one embodiment, the hot-water extraction may be performed by adding 1 L of distilled water per 100 g of coffee powder, applying heat at 121° C. for 15 minutes, and naturally cooling a temperature of the extract to room temperature.

The solvent may be one or more selected from distilled water, ethanol, methanol, hexane, chloroform, methylene chloride, ethyl acetate, and diethylene glycol monoethyl ether.

After heating, extracting and filtrating are performed, all solvents may be removed by performing concentration under reduced pressure, and the concentrate may be freeze-dried to obtain an extract.

As the coffee extract, a coffee hot-water extract obtained by heating the roasted coffee beans with distilled water, extracting the roasted coffee beans, and concentrating the resultant under reduced pressure may be used.

In addition, a coffee alcohol extract obtained by heating the roasted coffee beans with alcohol, extracting the roasted coffee beans, and concentrating the resultant under reduced pressure may be used.

Moreover, a water-soluble coffee extract obtained by heating the roasted coffee beans with alcohol, extracting the roasted coffee beans, and concentrating the resultant under reduced pressure to remove alcohol, and then dissolving the resultant in distilled water may be used.

The water-soluble coffee extract is well dissolved in water and has low viscosity, so that it is easy to process, and a content of some active ingredients may increase through a dissolving process.

The water-soluble coffee extract may be used as it is or after removing distilled water by concentration under reduced pressure.

According to the present invention, one or more selected from the coffee hot-water extract, the coffee alcohol extract, and the water-soluble extract may be used as the coffee extract, and the coffee extract is preferably the coffee hot-water extract.

In addition, according to the present invention, the coffee hot-water extract and the water-soluble coffee extract may be simultaneously used as the coffee extract, in which a weight ratio of the coffee hot-water extract and the water-soluble coffee extract is preferably 60 to 80:20 to 40.

According to the present invention, as the coffee hot-water extract, a hot-water extract of coffee beans roasted at 200° C. to 250° C. for 30 seconds to 18 minutes may be used.

In addition, at least one of a hot-water extract of coffee beans (Mild) roasted at 200° C. to 250° C. for 30 seconds to 2 minutes, a hot-water extract of coffee beans (Medium) roasted at 200° C. to 250° C. for 3 to 6 minutes, a hot-water extract of coffee beans (Med-dark) roasted at 200° C. to 250° C. for 7 to 10 minutes, and a hot-water extract of coffee beans (Dark) roasted at 200° C. to 250° C. for 12 to 18 minutes may be used.

According to the present invention, as the coffee hot-water extract, it is preferable to use the hot-water extract of coffee beans (Mild) roasted at 200° C. to 250° C. for 30 seconds to 2 minutes, and it is most preferable to use the hot-water extract of coffee beans roasted at 220° C. for 1 minute.

In addition, according to the present invention, the hot-water extract of coffee beans (Mild) roasted at 200° C. to 250° C. for 30 seconds to 2 minutes and the hot-water extract of coffee beans (Medium) roasted at 200° C. to 250° C. for 3 to 6 minutes may be used as the roasted coffee beans. In this case, a weight ratio of the hot-water extract and the hot-water extract of Medium is preferably 60 to 80:20 to 40.

In addition, according to the present invention, the hot-water extract of coffee beans (Mild) roasted at 200° C. to 250° C. for 30 seconds to 2 minutes, the hot-water extract of coffee beans (Medium) roasted at 200° C. to 250° C. for 3 to 6 minutes, and the hot-water extract of coffee beans (Dark) roasted at 200° C. to 250° C. for 12 to 18 minutes may be used as the roasted coffee beans. In this case, a weight ratio of the hot-water extract Mild, the hot-water extract of Medium, and the hot-water extract of Dark is preferably 100:30 to 50:10 to 30.

In addition, the present invention relates to a pharmaceutical composition including a coffee extract as an active ingredient for preventing or treating diabetes.

The pharmaceutical composition of the present invention may further include a suitable carrier, excipient, and diluent commonly used in production of the pharmaceutical composition.

Examples of the carrier, the excipient, or the diluent may include lactose, texrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

The pharmaceutical composition of the present invention may be used by formulating the pharmaceutical composition in the form of oral formulations such as powders, granules, tablets, capsules, suspensions, emulsions, syrups, and aerosols, and in the form of external preparations, suppositories, and sterile injectable solutions.

In addition, the present invention relates to a food composition including a coffee extract as an active ingredient for preventing or alleviating diabetes.

The food may be in the form of various foods, candies, beverages, gum, tea, vitamin complexes, health functional foods, and the like, and may be provided in the form of powders, granules, tablets, capsules, beverages, and the like.

Hereinafter, the present invention will be described in detail through examples. The following examples are merely exemplified for the practice of the present invention, and the contents of the present invention are not limited by the following examples.

### (Example 1) In-Vitro Antidiabetic Activity of Coffee Extract

### (a) Rat Intestinal α-Glucosidase Inhibition Assay

Enzyme: rat small intestinal acetone powder
Substrate: PNP-glycoside (pNPG, p-Nitrophenyl α-Dglucopyranoside)

150 mg of rat small intestine acetone powder was added to 9 ml of 0.1 M sodium phosphate buffer (pH 6.9; including 0.9% sodium chloride), ultrasonicated 12 times in an ice water bath for 30 seconds, and then centrifuged at 13,000 rpm and 4° C. for 3 minutes. After centrifugation, a supernatant was collected and used in rat intestinal α-glucosidase inhibition assay.

100 μl of a rat α-glucosidase solution and 50 μl of a sample solution were added and reacted at 37° C. for 10 minutes, and then 50 μl of 5 mM pNPG solution was added thereto to react the mixture at 37° C. for 10 minutes.

After completion of the reaction, inhibitory activity against rat intestinal α-glucosidase was analyzed by measuring absorbance at 405 nm by using an ELISA reader.

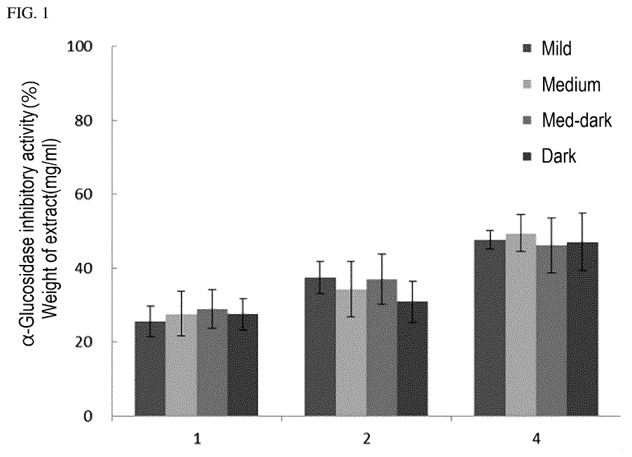

FIG. 1 shows inhibitory activity of a coffee extract against rat intestinal α-glucosidase.

In this case, Mild refers to a hot-water extract of coffee beans roasted at 220° C. for 1 minute, Medium refers to a hot-water extract of coffee beans roasted at 220° C. for 5 minutes, Med-dark refers to a hot-water extract of coffee beans roasted at 220° C. for 8 minutes, and Dark is a hot-water extract of coffee beans roasted at 220° C. for 15 minutes.

The hot-water extraction may be performed by adding 1 L of distilled water per 100 g of coffee powder, applying heat at 121° C. for 15 minutes, and naturally cooling a temperature of the extract to room temperature.

The sample solution used in the experiment was prepared as follows. The hot-water extract was centrifuged (8000 rpm, 30 min), the supernatant was filtered through a filter paper (90 mm/Whatman™), and the filtrate was concentrated to 60 to 150 ml and freeze-dried to produce extract powder. 100 mg of powder was mixed with 1 L of water to prepare a sample solution.

As a concentration of the coffee hot-water extract increased, the inhibitory activity against rat intestinal α-glucosidase increased.

It can be seen that the inhibitory activity of the hot-water extract of roasted coffee beans is increased in dependence on the concentration, and the inhibitory activity of the hot-water extract of Mild and Medium is the most excellent.

The following Table 1 shows the 50% inhibitory activity concentration (IC₅₀) for rat intestinal α-glucosidase of the coffee hot-water extract.

TABLE 1

| | The half maximal inhibitory |
|---|---|
| | concentration (IC₅₀) (mg/ml) |
| Hot-water extract of Mild | 4.17 |
| Hot-water extract of Medium | 4.11 |
| Hot-water extract of Med-dark | 4.38 |
| Hot-water extract of Dark | 4.43 |

The following Table 2 shows 50% inhibitory activity concentration (IC₅₀) for rat intestinal a-glucosidase when the coffee hot-water extracts are mixed and used.

TABLE 2

| Hot-water extract of Mild:Hot-water | |
|---|---|
| extract of Medium | The half maximal inhibitory |
| (Weight ratio) | concentration (IC₅₀) (mg/ml) |
| 100:0 | 4.17 |
| 90:10 | 4.15 |
| 70:30 | 3.74 |
| 50:50 | 4.22 |
| 0:100 | 4.11 |

When a weight ratio of the hot-water extract of Mild and the hot-water extract of Medium was 70:30, the 50% inhibitory activity concentration (IC₅₀) showed the lowest value.

According to the present invention, as the coffee hot-water extract, a coffee hot-water extract may be used by mixing the hot-water extract of coffee beans (Mild) roasted at 200° C. to 250° C. for 30 seconds to 2 minutes and the hot-water extract of coffee beans (Medium) roasted at 200° C. to 250° C. for 3 to 6 minutes, and in this case, a weight ratio of the hot-water extract of Mild and the hot-water extract of Medium is preferably 60 to 80:40 to 20.

The following Table 3 shows 50% inhibitory activity concentration (IC₅₀) for rat intestinal a-glucosidase when the coffee hot-water extracts are mixed and used.

TABLE 3

| Hot-water extract of Mild:Hot-water | |
|---|---|
| extract of Medium:Hot-water | The half maximal |
| extract of Dark | inhibitory concentration |
| (Weight ratio) | (IC₅₀) (mg/ml) |
| 100:40:5 | 4.19 |
| 100:40:20 | 3.79 |
| 100:40:40 | 4.26 |

When a weight ratio of the hot-water extract of Mild, the hot-water extract of Medium, and the hot-water extract of Dark was 100:40:20, the 50% inhibitory activity concentration (IC₅₀) showed the lowest value.

According to the present invention, as the coffee hot-water extract, a coffee hot-water extract may be used by mixing the hot-water extract of coffee beans (Mild) roasted at 200° C. to 250° C. for 30 seconds to 2 minutes, the hot-water extract of coffee beans (Medium) roasted at 200° C. to 250° C. for 12 to 18 minutes, and the hot-water extract of coffee beans (Dark) roasted at 200° C. to 250° C. for 12 to 18 minutes, and in this case, a weight ratio of the hot-water extract of Mild, the hot-water extract of Medium, and the hot-water extract of Dark is preferably 100:30 to 50:10 to 30.

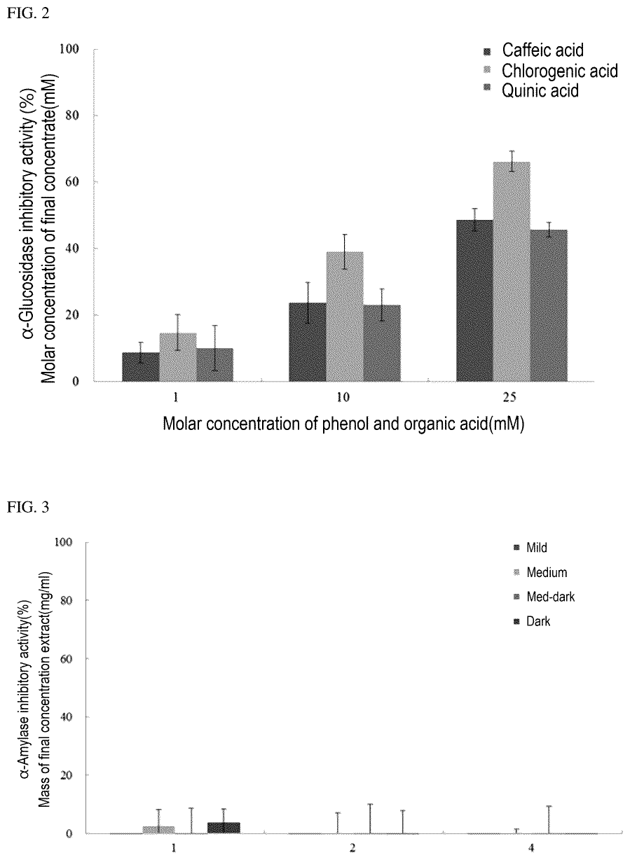

FIG. 2 shows inhibitory activity of caffeic acid, chlorogenic acid, and quinic acid against rat intestinal α-glucosidase.

It can be seen that inhibitory activity of caffeic acid, chlorogenic acid, and quinic acid against α-glucosidase increases in dependence on the concentration, and the chlorogenic acid inhibitory activity is the most excellent.

### (b) Porcine Pancreatic α-Amylase Inhibition Assay

Enzyme: porcine pancreatic α-amylase
Substrate: 1% starch solution in 0.02 M sodium phosphate buffer (pH 6.9)
Coloring Reagent: 3,5-dinitrosalicylic acid solution (DNS) in 2 M NaOH with 30% sodium potassium tartrate tetrahydrate

200 μl of a sample solution was added to 300 μl of a porcine pancreatic α-amylase solution having a concentration of 10 U, which is dissolved in a 0.02 M sodium phosphate buffer (pH 6.9; including 0.006 M sodium chloride), and incubated at 25° C. for 10 minutes.

500 μl of a 1% starch solution, which was pre-cultured at 25° C. for 10 minutes, was added to the solution, and the mixture was reacted at 25° C. for 10 minutes.

1 ml of a 1% DNS solution dissolved in 30% Rochelle salt was added and treated in boiling water bath for 5 minutes and cooled to room temperature, and 10 ml of distilled water was added to the resultant.

The absorbance of a reaction solution of the DNS solution and sugar decomposed from the substrate by α-amylase was measured at 540 nm by using an ELISA reader.

FIG. 3 shows inhibitory activity of a coffee extract against porcine pancreatic α-amylase.

The hot-water extract of roasted coffee beans does not exhibit inhibitory activity against a-amylase, and these results show the possibility that indigestion due to excessive inhibition of amylase, gas production due to starch fermentation in the colon due to introduction of starch that is not decomposed and absorbed in the small intestine, and the like may be alleviated.

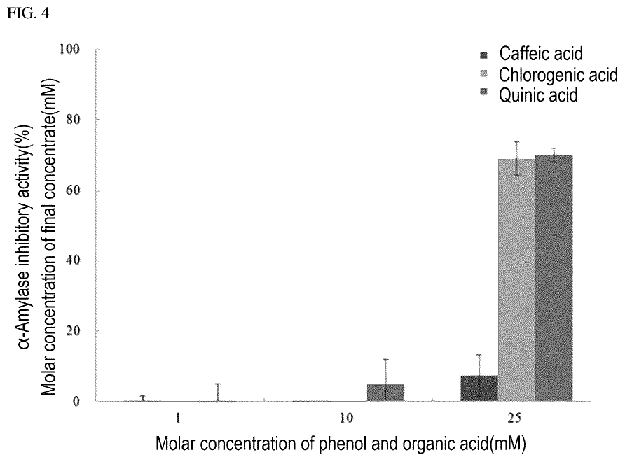

FIG. 4 shows inhibitory activity of caffeic acid, chlorogenic acid, and quinic acid against porcine pancreatic α-amylase.

It can be seen that chlorogenic acid and quinic acid have excellent inhibitory activity against α-amylase at a concentration of 25 mM/ml.

### (c) Content of Phenol-Based Compound

1 ml of a sample solution, 1 ml of 95% ethanol, 5 ml of distilled water, and 0.5 ml of 50% Folin-Ciocalteu phenol reagent were added to a test tube and reacted at 25° C. for 5 minutes, and then 31 ml of 5% Na₂CO was added and stored in a dark room for 1 hour.

After vortex, the absorbance of the resultant was measured at 725 nm by using a spectrophotometer (Shimadzu; UV160A).

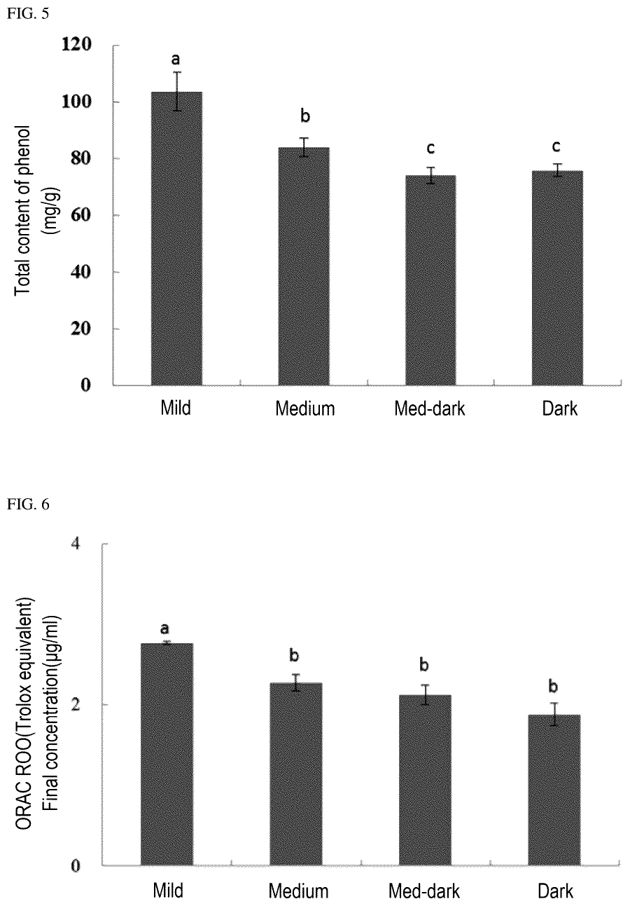

FIG. 5 shows a content of a phenol-based compound in a coffee extract.

It can be seen that a content of the phenol-based compound is the highest in the hot-water extract of Mild.

Therefore, a polyphenol component contains the most abundant hot-water extract of roasted coffee bean (Mild).

### (d) Antioxidant Activity

A rate of decrease in absorbance of fluorescein due to generation and disappearance of peroxy radical causing oxidation in vivo was measured. 2,2′-azobis (2-aminopropane) dihydrochloride (AAPH, 20 mM) was used for the generation of the peroxide radical, and SpetraMax®i3 (MolecularDevices) was used for measuring instrument, and electrons were set to be absorbed at 485 nm and emitted at 538 nm. A fluorescein value was relatively shown compared to an initial value, and an ORAC value was shown as μM Trolox equivalent per 1 g.

FIG. 6 shows antioxidant activity of a coffee extract.

It can be seen that the hot-water extract of Mild shows the most excellent antioxidant activity.

### (Example 2) Postprandial Blood Sugar Control Action by In-Vivo Test of Coffee Extract

### (a) Experimental Animal

An SD rat, a 5-week-old male, was purchased from RAONBIO, bred in an animal breeding room, and only healthy animals at 6 weeks of age were selected and used in the experiment.

### (b) Evaluation of Blood Sugar Rise Suppression Action

After fasting the experimental animals for 24 hours or longer before the experiment, the coffee extract was administered at a concentration of 0.5 g/kg to 2 g/kg body weight of sucrose or starch, and the sample was orally administered using a zonde for oral administration.

The administration group was 6 groups, 6 per each group. The blood was collected from the rat tail vein at 30, 60 and 120 minutes after oral administration, and changes in blood sugar concentration in the venous blood were measured using a blood sugar meter (Caresens II).

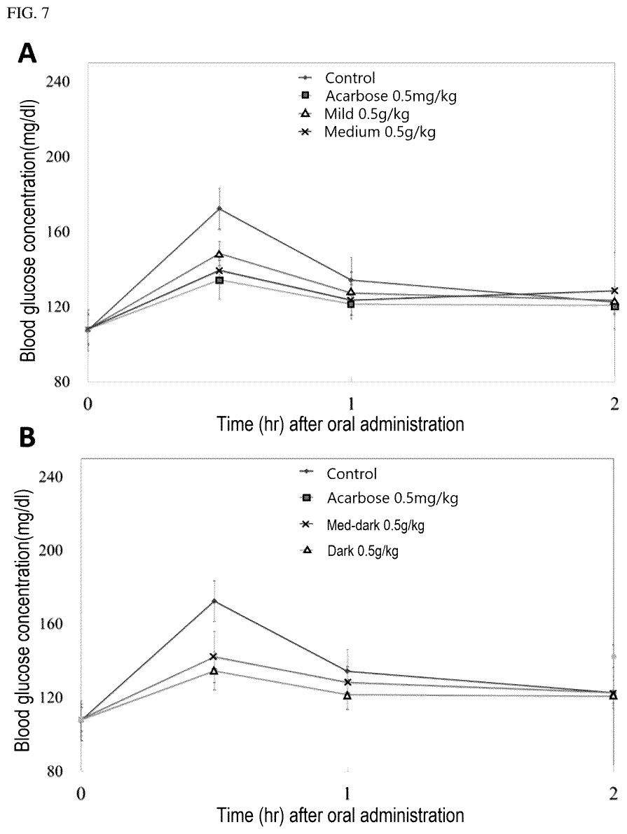

FIG. 7 shows inhibition action on postprandial blood sugar rise of a coffee extract by sucrose.

The blood sugar increased to 172.23±11.03 mg/dl 30 minutes after administration for a control to which only sucrose was administered, but the postprandial blood sugar levels decreased to 148.41±6.55 mg/dl for Mild, 144.66±6.45 mg/dl for Medium, 143.91±13.94 mg/dl for Med-dark, and 145.83±6.97 mg/dl for Dark.

From 30 minutes to 2 hours, the blood sugar rapidly decreased to 134.16±12.01 mg/dl and 122.5±6.96 mg/dl for the control, and the postprandial blood sugar decreased to 127.25±11.23 mg/dl and 123.41±7.21 mg/dl for Mild, decreased to 123.5±8.28 mg/dl and 128.58±20.32 mg/dl for Medium, decreased to 128.16±8.55 mg/dl and 122.66±8.63 mg/dl for Med-dark, and decreased to 131.83±7.43 mg/dl and 131.08±6.14 mg/dl for Dark, as compared to the control.

Therefore, the coffee hot-water extract has an effect of suppressing postprandial blood sugar rise by inhibiting absorption in an upper part of the small intestinal.

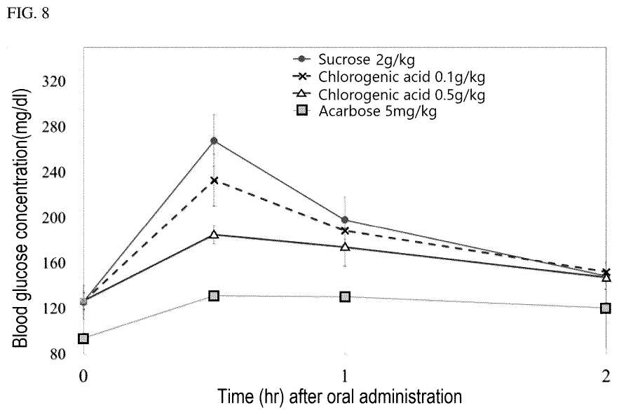

FIG. 8 is a view confirming that chlorogenic acid suppresses the postprandial blood sugar rise after sucrose intake.

The blood sugar increased to 267.75±22.65 mg/dl 30 minutes after administration for the control to which only sucrose was administered, but the postprandial blood sugar levels decreased to 184.93±8.24 mg/dl for 0.1 g/kg of chlorogenic acid and 131.29±14.47 mg/dl for 0.5 g/kg of chlorogenic acid.

From 30 minutes to 2 hours, the blood sugar rapidly decreased to 198.18±20.36 mg/dl and 148.56±11.62 mg/dl for the control, and the postprandial blood sugar decreased to 174.00±16.67 mg/dl and 147.38±10.86 mg/dl for 0.1 g/kg of chlorogenic acid and 130.64±10.90 mg/dl and 120.69±13.77 mg/dl for 0.5 g/kg of chlorogenic acid, as compared to the control.

Therefore, chlorogenic acid has an effect of suppressing postprandial blood sugar rise by inhibiting absorption in an upper part of the small intestinal in dependence on the concentration.

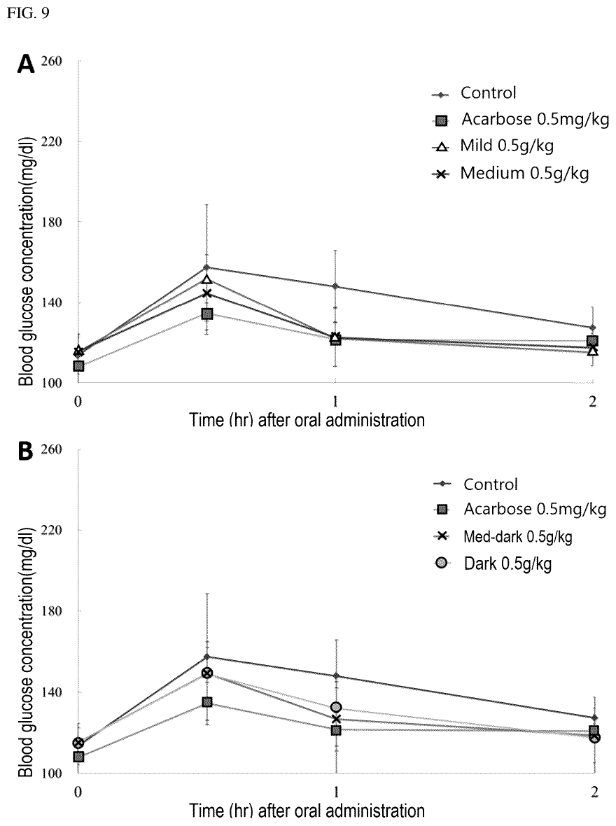

FIG. 9 is a view confirming that the coffee hot-water extract suppresses the postprandial blood sugar rise after starch intake.

The blood sugar increased to 174.35±21.24 mg/dl 30 minutes after administration for the control to which only starch was administered, but the postprandial blood sugar levels decreased to 154.71±11.98 mg/dl for Mild, 144.5±13.89 mg/dl for Medium, 160.92±15.60 mg/dl for Med-dark, and 156.5±13.05 mg/dl for Dark.

From 30 minutes to 2 hours, the blood sugar slowly decreased to 147.92±17.80 mg/dl and 27.21±10.40 mg/dl for the control, and the postprandial blood sugar decreased to 121.92±15.40 mg/dl and 115±6.75 mg/dl for Mild, decreased to 122.5±14.39 mg/dl and 117.35±8.99 mg/dl for Medium, decreased to 126.64±15.48 mg/dl and 118.64±13.53 mg/dl for Med-dark, and decreased to 132±12.96 mg/dl and 117.55±11.47 mg/dl for Dark, as compared to the control.

Therefore, the coffee hot-water extract has an effect of suppressing postprandial blood sugar rise by inhibiting absorption in an upper part of the small intestinal.

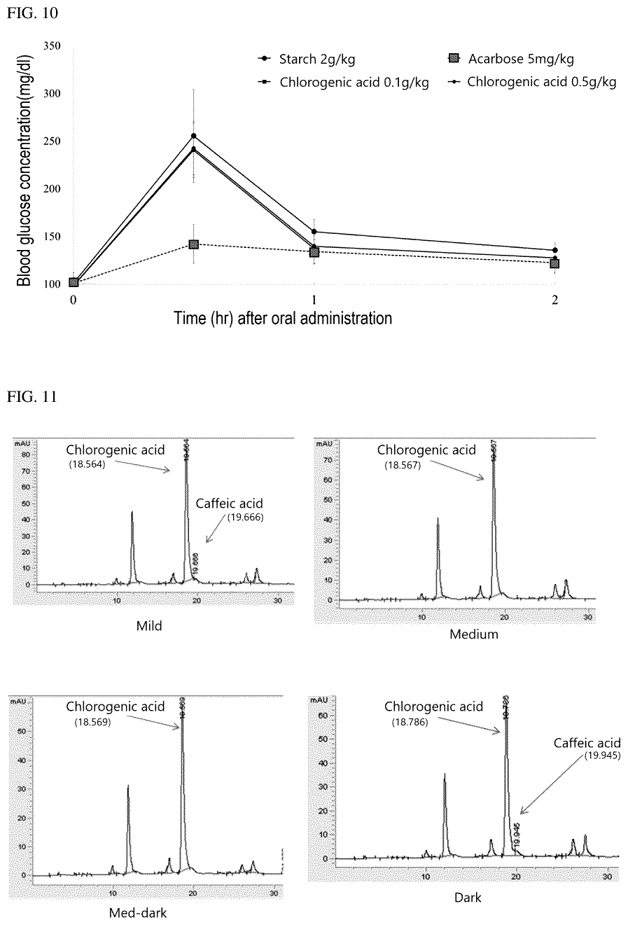

FIG. 10 is a view confirming that chlorogenic acid suppresses the postprandial blood sugar rise after starch intake.

The blood sugar increased to 255.58±51.1 mg/dl 30 minutes after administration for the control to which only starch was administered, but the blood sugar decreased to 239.00±25.86 mg/dl for 0.1 g/kg of chlorogenic acid and 225.17±18.37 mg/dl for 0.5 g/kg of chlorogenic acid, which was similar to the control.

From 30 minutes to 2 hours, the blood sugar rapidly decreased to 156.08±12.92 mg/dl and 135.58±7.79 mg/dl for the control, and the blood sugar decreased to 137.33±10.46 mg/dl and 126.08±10.75 mg/dl for 0.1 g/kg of chlorogenic acid and 137.23±9.58 mg/dl and 126.46±12.1 mg/dl for 0.5 g/kg of chlorogenic acid, which was similar to the control.

### (Example 3) HPLC Measurement of Coffee Hot-Water Extract

FIG. 11 shows HPLC results of a coffee extract.

In a hot-water extract of coffee waste, a small amount of chlorogenic acid was detected at about 26 mAU, and caffeic acid was not detected. (HPLC results omitted)

On the other hand, the hot-water extract of Mild coffee includes chlorogenic acid and caffeic acid, and a content of chlorogenic acid is the highest compared to other coffee hot-water extracts. Specifically, in the hot-water extract of Mild coffee, about 85 mAU of chlorogenic acid and about 3 mAU of caffeic acid were detected.

The hot-water extract of Medium coffee and the hot-coffee extract of Medium-dark coffee include chlorogenic acid, and the hot-water extract of Dark coffee includes chlorogenic acid and caffeic acid. Specifically, in the hot-water extract of Medium coffee, about 75 mAU of chlorogenic acid was detected, and caffeic acid was not detected. In the hot-water extract of Med-Dark coffee, about 59 mAU of chlorogenic acid was detected, and caffeic acid was not detected. In the hot-water extract of Dark coffee, about 67 mAU of chlorogenic acid and about 3 mAU of caffeic acid were detected.

1. A composition comprising a coffee extract as an active ingredient for suppressing postprandial blood sugar rise.

2. The composition of claim 1, wherein the coffee extract is a hot-water extract of roasted coffee beans.

3. The composition of claim 2, wherein the coffee extract includes a hot-water extract of coffee beans roasted at 200° C. to 250° C. for 30 seconds to 2 minutes and a hot-water extract of coffee beans roasted at 200° C. to 250° C. for 3 to 6 minutes.

4. A pharmaceutical composition comprising a coffee extract as an active ingredient for preventing or treating diabetes.

5. A food composition comprising a coffee extract as an active ingredient for preventing or alleviating diabetes.
